(19) United States
(12) Patent Application Publication
(10) Pub. No.: US 2023/0293268 A1
(43) Pub. Date: Sep. 21, 2023

(54) DEVICE AND METHOD FOR BIOELECTRIC STIMULATION ACCELERATING BONE INTEGRATION INTO IMPLANT HEALING

(71) Applicant: Leonhardt Ventures LLC, Mission Viejo, CA (US)

(72) Inventors: Howard J. Leonhardt, Mission Viejo, CA (US); Tom Newman, Newport Beach, CA (US)

(21) Appl. No.: 18/186,811

(22) Filed: Mar. 20, 2023

Related U.S. Application Data
(60) Provisional application No. 63/321,222, filed on Mar. 18, 2022.

(51) Int. Cl. A61C 8/02; A61N 1/32

(52) U.S. Cl. CPC A61C 8/0007; A61N 1/326

(57) ABSTRACT

Described are a device, system, and method for bioelectric stimulation to accelerate bone integration of an implant and/or bone graft in order to aid healing. Specifically described are devices and methods for enhancing bone integration of a body implant (e.g., a dental implant) or bone graft by modulating (e.g., stimulating) the controlled expression and/or release of selected proteins, which proteins promote osteogenesis and osseointegration via specific bioelectric signals delivered via, e.g., a specialized mouthpiece, electrodes, and/or wireless means. The description particularly relates to the acceleration of the healing of dental implants and/or bone grafts via promoting bone osteointegration more quickly with bioelectric stimulation.

### CROSS-REFERENCE TO RELATED APPLICATION

The application claims the benefit of U.S. Provisional Application Ser. No. 63/321,222 filed on Mar. 18, 2022, the contents of which are incorporated herein by this reference.

### TECHNICAL FIELD

The application relates to medical and dental treatments generally, and more particularly to a device and associated methods for bioelectric stimulation of patient cellular tissue to accelerate bone integration of an implant and/or bone graft and associated healing. Specifically, this application relates to devices and methods for enhancing bone integration of a body implant (e.g., a dental implant), an implant with autograft or allograft bone, and/or bone graft by modulating (e.g., stimulating) the controlled expression and/or release of selected proteins, which proteins promote osteogenesis and osseointegration via specific bioelectric signals from a biostimulator delivered by way of, e.g., a specialized mouthpiece, electrodes, and/or wireless means. The application particularly relates to the acceleration of the healing of dental implants via promoting bone osteointegration more quickly with bioelectric stimulation.

### BACKGROUND

In implant dentistry, an artificial root is placed into a subject's jawbone to hold a replacement tooth (crown). The two-part procedure begins with screwing a titanium (or other metal or material such as ceramic) “root” into the jawbone and waiting three to six months (sometimes even a year) until the bone grows firmly around it—called “osseointegration.” A healing abutment, or cap, is placed on top of the implant to help the gum heal, and then it's replaced with a regular cap in which the crown is connected to the implant.

Attempts have been made to utilize an electromagnetic field to stimulate osteogenesis and osseointegration.

For example, U.S. Pat. No. 5,292,252 to Nickerson et al., the contents of which are incorporated herein by this reference, discloses a stimulator healing cap for enhancing the growth of bone cells and bone tissue surrounding a dental implant. The stimulator healing cap includes a top cap portion containing a direct current source, and a threaded portion which attaches to the implant in the same way as a cover screw. In one embodiment, the current source is coupled to a coil which surrounds a longitudinal core creating an electromagnetic field around the implant and thus in the surrounding bone tissue.

According to U.S. Pat. No. 9,327,115 to Neuman et al., the contents of which are incorporated herein by this reference, the electromagnetic field generated by the current source of Nickerson et al. is a static magnetic field (SMF), and the stimulation of bone growth by a SMF has not yet been definitively established. Also according to U.S. Pat. No. 9,327,115, another disadvantage of this healing cap is that the constantly operating battery that can be housed within its small internal volume is of an insufficient capacity for the required healing period.

Also, U.S. Pat. No. 6,034,295 to Rehberg et al., the contents of which are incorporated herein by this reference, discloses an implantable device, including a dental implant, formed with an internal cavity into which the bone tissue that surrounds the implanted device is intended to grow. The device is provided with at least two electrodes, one of which is located within the cavity and spaced apart from the inside of the body that forms the cavity, and a second electrode when the body is made of an electrically conductive material, which together with the internal electrode, forms a kind of coaxial structure for generating a low-frequency alternating current for promoting tissue growth. Also according to U.S. Pat. No. 9,327,115, the electric field generated by electrodes such as those of Rehberg will not propagate beyond the outermost electrode, and is therefore incapable of stimulating osteogenesis in damaged or osteoporotic tissue outwardly spaced from the implant.

In 2012, a University of Maryland research team reported at the 27th Annual Meeting of the Academy of Osseointegration reported that electrical stimulation expedites bone graft healing and can increase the predictability and contribute to the overall success of this option for patients who lack the bone density required for dental implants. “Electrical stimulation speeds bone graft healing and can increase success of grafting for dental implants” infra. In the study, bipolar platinum stimulated electrodes were overlaid on the center of a graft in adult male rats. They received electrical stimulation three times a day for 10 days. After six weeks, the grafted areas and surrounding bone were harvested. Animals that received electrical stimulation had approximately eight-fold more new bone (3.81 (3.6)%; p=0.034) compared to the control group (0.47 (0.52)%). The amount of remaining graft material in the control group was significantly higher, and no significant difference was found in the amount of connective tissue.

While all steps in the right direction, the application of particularly selected bioelectric signals to accelerate osseointegration would be a significant improvement in the art.

### BRIEF SUMMARY

Described is a device for stimulating accelerated and more complete bone integration and healing of a body implant comprising a bioelectric stimulator programmed to deliver precise bioelectric signals on demand to control specific osteogenesis and osseointegration promoting proteins such as OPG via electrodes or wireless energy means. Also described is a method for stimulating accelerated and more complete bone integration and healing of a body implant comprising delivering precise bioelectric signaling sequences on demand to control specific osteogenesis and osseointegration promoting proteins such as OPG via electrodes or wireless energy means.

Specifically described are devices and methods utilized to enhance bone integration into a body implant by stimulating the precise controlled release of osteogenesis and osseointegration promoting proteins via specific bioelectric signaling sequences. Bioelectric signals may be delivered via a specialized mouthpiece or via wireless means.

In certain embodiments, described is a bioelectric signal applicator mouthpiece connected to an external bioelectric stimulator that delivers specific bioelectric signals for control of protein expressions designed to accelerate dental implant healing and osseointegration (bone in growth), and to reduce associated patient pain and discomfort. The system and method decrease the required time for a dental implant to be fully loaded in normal activities such as chewing food without pain.

Described are devices and associated methods for accelerating the healing, especially time to stability, and reducing the pain and discomfort associated with implantable devices, such as dental implants, via application of bioelectric signals to modulate (e.g., upregulate) the expression of various protein(s) by the patient.

Described are a device, system, and method for increasing the rate of, for example, oral tissue healing via the application of selected bioelectric signals to the tissue to control protein expression by the tissue.

The described method is particularly useful for use in a patient following the placement of at least one dental implant in the patient's oral tissue. Such a method typically comprises applying or administering to the patient's oral tissue at least one bioelectric signal from an electrical stimulator apparatus (a “biostimulator”). The biostimulator apparatus is preferably further associated with (e.g., electrically connected to) a device (e.g., a mouthpiece) placed within the patient's mouth that assists in delivering the bioelectric signals to the target tissues.

The described method may further include operatively controlling, during the administration of the bioelectric signals and via an electronic circuit of the biostimulator, an operational state of the biostimulator, a frequency of the bioelectric stimulation, voltage intensity of the bioelectric signals from the biostimulator, a amperage flow of the bioelectric signals through the target tissue from the biostimulator, and duration of bioelectric stimulation from the biostimulator.

In certain embodiments, the biostimulator is able to deliver in sequence a plurality of bioelectric signals in succession. In certain embodiments, the administration of bioelectric signals occurs within twenty-four hours of the placement of the at least one dental implant. In certain embodiments, the administration of the bioelectric signals occurs within six hours of the placement of the at least one dental implant. In certain embodiments, the administration of the bioelectric signals occurs within one hour of the placement of the at least one dental implant. In certain embodiments, the administration of bioelectric signals occurs for from about one minute to about 60 minutes.

In certain embodiments, the mouthpiece displaces at least a portion of the patient's buccal tissue away from the patient's alveolar soft tissue when the mouthpiece is disposed within the mouth.

In certain embodiments, the administration of bioelectric signals to the patient's oral tissue includes administering bioelectric signals to the patient's alveolar soft tissue, and the apparatus includes a reflective material coupled to the mouthpiece configured to direct the bioelectric signals from the biostimulator to the alveolar soft tissue.

In certain embodiments (e.g., for treating more severe cases), described is a treatment that combines the application of bioelectric signals with the administration of biologics (such as platelet rich plasma (PRP) or platelet rich fibrin (PRF)) for more rapid healing. For example, bioelectric stimulation may be combined with a biologics composition to accelerate healing including “bioelectric PRF” and regenerative fluid, derived from amniotic fluid and exosomes. See, e.g., US Patent Application Publication US 20200000709 A1 to Leonhardt et al. (Jan. 2, 2020) for “Combination of bioelectrical stimulator and platelet-rich fibrin for accelerated healing and regeneration.” In certain such embodiments, the benefits of precise bioelectric signaling controlled protein expressions are combined with repeat delivery of biologics via a slow infusion re-fillable micro infusion pump placed on top of the dental implants. This micro pump may be re-filled daily or weekly with an implant healing acceleration composition comprising a combination of components, such as PRP, tooth pulp stem cells, bioelectric PRF, amniotic fluid, micro RNA gel, bone matrix, selected growth factors such as BMPs, nutrient hydrogel, oxygenated nanoparticles, and bone matrix.

In certain embodiments, the bioelectric signals are combined with a bone graft (allograft, autograft, man-made, or xenograft) to make the host bone and graft incorporate faster and stronger. Such embodiments are particularly useful in orthopedics, spine, trauma, and dental applications.

Described are methods for healing bone grafts more rapidly by applying bioelectric signals that upregulate expression of BMP9, osteoprotegerin (OPG), Klotho, IGF1, BMP3, and other signals. Such methods not only have dental applications, but also use with orthopedic, spine fusion and other applications involving bone grafts.

The described therapy improves bone formation (osteogenesis), accelerates the healing process via osseointegration, reduces patient pain and discomfort; increases bone to implant connection (BIC), increases trabecular bone volume density, reduces the time healing time, enables quicker placement of the crown, and reduces the need for repeat/repair surgeries. The described therapy is also believed to be a suitable alternative for patients who may not otherwise be considered good candidates for implants, having poor bone quality from underlying diabetes, autoimmune disorders, as well as heavy smokers and drinkers.

### BRIEF DESCRIPTION OF THE DRAWINGS

FIG. 1 depicts a bioelectric stimulator.

FIG. 2 depicts a mouthpiece, which connects to a bioelectric stimulator, having electrodes for applying bioelectric signals to the patient's gums and treated area.

FIG. 3 depicts a bioelectric stimulator and associated mouthpiece and electrodes for applying bioelectric signals to the patient's oral cavity.

FIG. 4 depicts a dental implant.

FIG. 5 is a graph depicting cumulative results of OPG expression for specific bioelectric signal application protocols as applied to osteoblasts.

### DETAILED DESCRIPTION

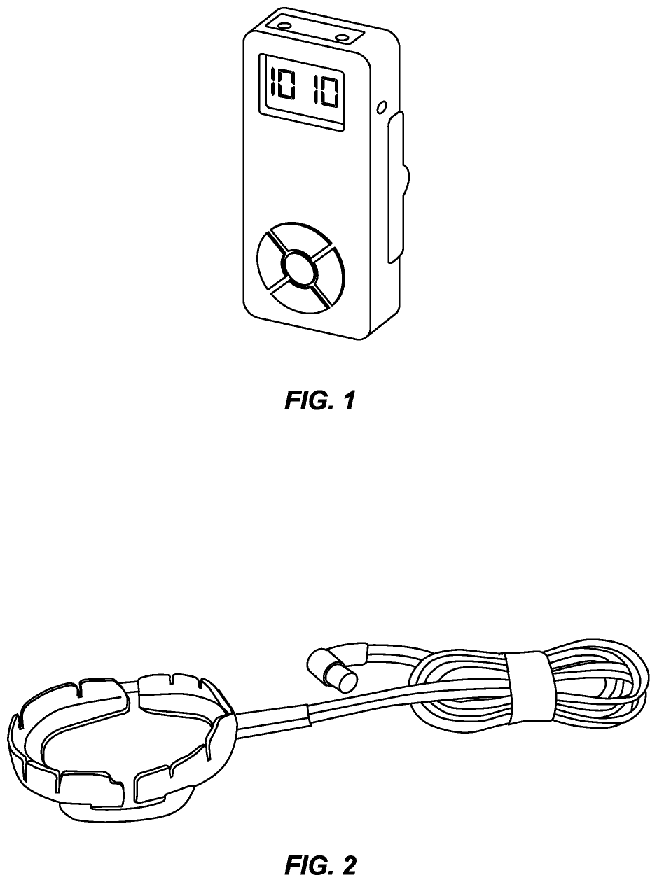

Referring now to FIG. 1, depicted is a biostimulator for use in the treatment of a, for example, human subject. A micro voltage signal generator for use herein may be produced utilizing the same techniques to produce a standard heart pacemaker well known to a person of ordinary skill in the art. An exemplary microvoltage generator is available from Mettler Electronics Corp. of Anaheim, California, US or HTM Electrônica of Amparo, BR. The leading pacemaker manufacturers are Medtronic, Boston Scientific Guidant, Abbott St. Jude, BioTronik and Sorin Biomedica.

Construction of the electric signal generators and pacemakers, are known in the art and can be obtained from OEM suppliers as well as their accompanying chargers and programmers. The electric signal generators are programmed to produce specific bioelectric signals to lead to specific protein expressions at precisely the right time for, e.g., optimal treatment or for tissue regeneration.

Described is a system for selectively administering electrical stimuli to a patient undergoing an implant and/or bone graft procedure, the system comprising: a programmable source of variable frequency, variable amplitude electrical signals, wherein the frequency is capable of being varied within a range of approximately 0.1 to 970 Hertz and the amplitude is capable of being varied within a range of approximately 20 to 400 microamps; and a plurality of electrodes to connect the source to a patient, wherein the variable frequency, variable amplitude electrical signals are applied as electrical stimuli to a region proximate to, e.g., the mouth of the patient to perform a predetermined protocol.

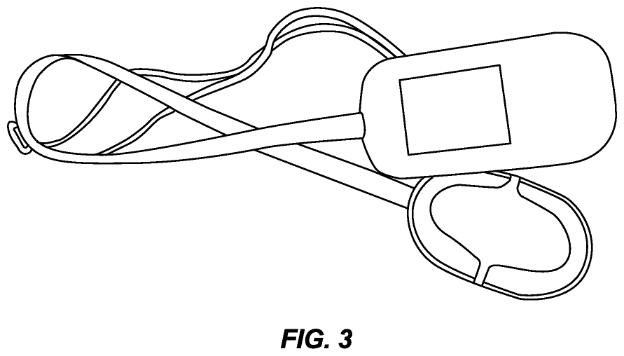

In certain embodiments, the biostimulator is electrically connected via wires to, for example, electrodes, which electrodes contact or are positioned for contact to the implant or to the subject's cellular tissue for application of at least one bioelectric signal. In certain embodiments, the electrodes are appropriately placed in a mouthpiece (e.g., FIG. 2) and connected to the biostimulator as shown in FIG. 3. The mouthpiece is typically made of a biocompatible flexible material and contains the leads connecting the electrodes with the biostimulator. The mouthpiece is sized to fit as comfortably as possible in the patient's mouth.

The bioelectric stimulator is programmed to produce particular bioelectric signals, such as those disclosed in U.S. Pat. No. 10,960,206 to Leonhardt et al. for “Bioelectric Stimulator” (Mar. 20, 2021), the contents of the entirety of which are incorporated herein by this reference. Described therein are bioelectric signals to induce expression by cellular tissue of osteoprotegerin or “OPG,” RANKL, SDF-1, PDGF, a signal for stem cell homing, PDGF, different signals for stem cell proliferation, activin-B, EGF, IGF-1, tropoelastin, VEGF, follistatin, HGF, and any combination thereof.

The application of the bioelectric signals aids in the rapid accelerated healing in implant dentistry. The described combinations of bioelectric and biologics therapy keys on highly precise controlled protein expressions, such as OPG, SDF1, PDGF, VEGF, and IGF1, providing a treatment that activates, amplifies, extends, enhances and accelerates the natural healing process.

Particularly preferred proteins for use herein are OPG, Klotho, RANKL, IGF-1, a combination of bioelectrics and biologics for accelerated healing, and PRF.

Controlled release of OPG promotes osseointegration (bone in growth). SDF1 and PDGF promote stem cell homing and healing. The system promotes rapid regeneration of tissues.

For example, as described in the incorporated U.S. Pat. No. 10,960,206 to Leonhardt et al., the cellular expression of stromal cell-derived factor 1 (“SDF1,” also known as CXCL12) (a stem cell recruiting signal) is upregulated by the application of the following bioelectric signal to a subject's cellular tissue: 30 Hz with a voltage of 3.5 mV, and successively alternating currents of 700 to 1500 picoamps for one minute, and again with 700 to 1500 picoamps for one minute and stimulated with current of 0.25 mA, pulse duration of 40 Hz, pulse width of 100 μs, and frequency of 100 Hz.

As also described in the incorporated U.S. Pat. No. 10,960,206, the cellular expression of platelet-derived growth factor (“PDGF”) is upregulated by the application of the following bioelectric signal to a subject's cellular tissue: 3 V/cm, 10 Hz, 2 μA (0.000002 amps), and pulse duration of 0.2 ms. Another bioelectric signal that upregulates expression of PDGF is 20 V/cm, 100 Hz, 0.25 mA (2.5e-7 amps), and pulse duration of 40 Hz, width of 100 μs. A third such bioelectric signal described in U.S. Pat. No. 10,960,206 is 20 V for 1 minute, 20 mV for 10 minutes, current of 0.25 mA, pulse duration of 40 Hz, pulse width of 100 μs, and frequency of 100 Hz for 5 minutes followed by 528 Hz for 3 minutes and 432 Hz for 3 minutes and 50 Hz for 3 minutes.

As also described in the incorporated U.S. Pat. No. 10,960,206, the cellular expression of vascular endothelial growth factor (VEGF) is upregulated by the application of the following bioelectric signal to a subject's cellular tissue: 3 V/cm, 10 Hz, 2 μA (0.000002 amps), and pulse duration of 0.2 ms. As described in U.S. Patent Application Ser. No. 63/215,841 (filed Jun. 28, 2021) to Leonhardt et al. for “Modulation of Vascular Endothelial Growth Factor (VEGF) and Pulse Width Utilization,” other bioelectric signals upregulate the expression of VEGF.

As further described in the incorporated U.S. Pat. No. 10,960,206, the cellular expression of insulin-like growth factor 1 (“IGF-1”) is upregulated by the application of the following bioelectric signal to a subject's cellular tissue: within 15%, 3 mV with a frequency of about 22 Hz, and a current of about 1 mA, followed by 3 mA.

As further described in the incorporated US Publication No. 2020/033079 A1, the cellular expression of osteoprotegerin (“OPG”) may be upregulated by the application of the following bioelectric signal to a subject's cellular tissue: range 3 mV to 5 mV at frequency range 1 to 3 MHz duration range 30 to 40 mW/cm² for a minimum of 20 to 45 minutes.

The bioelectric stimulator may be programmed to produce a bioelectric signal of, within 15%, a biphasic current of frequency 20 Hz and a 7.8 ms pulse duration and/or produce at least one bioelectric signal having a frequency selected from the group consisting of 5 Hz, 10 Hz, 20 Hz, 25 Hz, 50 Hz, 75 Hz, 100 Hz, 250 Hz, 500 Hz, 750 Hz, 2,500 Hz, 100,000 Hz, 500,000 Hz, and 1 MHz. As described in US Patent Application Publication US 2020-0289826-A1 to Leonhardt et al. (Sep. 17, 2020) for “Klotho Modulation,” and U.S. patent application Ser. No. 17/473,809 to Leonhardt, filed Sep. 13, 2021, application of such bioelectric signals to a subject's tissue upregulates expression of Klotho.

For pain relief and inflammation control, the bioelectric stimulator may be further programmed to produce a bioelectric signal of a continuous current of 10 μA (as measured at the cellular level), for 5 minutes, where the continuous current has a biphasic waveform, with a frequency of 50 Hz, which downregulates the expression of interleukin-10 (IL-10).

Hypoxia-inducible factor 1-alpha (“HIF1α”) expression can be upregulated by the application of the following bioelectric signal to the subject's tissue: 0.25 mA to 0.75 mA (3.0V), 80 to 100 Hz, 80 to 110 μs pulse width, square wave (voltage/amperage being measured at the level of the cell being stimulated). Upregulation of expression of 285% has been observed.

HIF1α can be downregulated by the application of the following bioelectric signal to the subject's tissue: 30 Hz, 3.5 mV in Retinal Pigment Epithelium (RPE—purchased from ATCC) cells for 30 minutes.

The pharmacologic activation of the HIF-1 complex can be desirable in ischemic and inflammatory disorders. In contrast, HIF-1 blockade may be beneficial to prevent tumor angiogenesis and tumor growth. See, e.g., Wig-Bill-gel “Hypoxia-Inducible Factor-1 (HIF-1): A Novel Transcription Factor in Immune Reactions” Journal Of Interferon & Cytokine Research 25:297-310 (2005). Hypoxia-inducible factor-1 alpha has further uses, such as for liver treatment, repair, and regeneration. Tajima et al. “HIF-1alpha is necessary to support gluconeogenesis during liver regeneration.” Biochem Biophys Res Commun. 2009 Oct. 2; 387(4):789-94. doi: 10.1016/j.bbrc.2009.07.115. Epub 2009 Jul. 28. PMID: 19643083; Nath et al. “Hypoxia and hypoxia inducible factors: diverse roles in liver diseases.” Hepatology (Baltimore, Md.) vol. 55, 2 (2012): 622-33. doi:10.1002/hep.25497; Lin et al. “Hypoxia-Inducible Factor 2 Alpha Is Essential for Hepatic Outgrowth and Functions via the Regulation of leg1 Transcription in the Zebrafish Embryo” PLoS ONE 9(7): e101980; doi.org/10.1371/journal.pone.0101980. It also has uses in preventing kidney injury (Bhatt et al. “MicroRNA-687 Induced by Hypoxia-Inducible Factor-1 Targets Phosphatase and Tensin Homolog in Renal Ischemia-Reperfusion Injury” JASN July 2015, 26 (7) 1588-1596; DOI: doi.org/10.1681/ASN.2014050463) and treating ischemic hearts (Guimaraes-Camboa, N. and Evans, S. “Redox Paradox: Can Hypoxia Heal Ischemic Hearts?” Developmental Cell 39(4): 392-394 (Nov. 21, 2016); DOI: doi.org/10.1016/j.devcel.2016.11.007 and Cerrada et al. “Hypoxia-inducible factor 1 alpha contributes to cardiac healing in mesenchymal stem cells-mediated cardiac repair” Stem Cells Dev. 2013 Feb. 1; 22(3):501-11. doi: 10.1089/scd.2012.0340. Epub 2012 Sep. 14. PMID: 22873764).

In certain embodiments, the described methods include use of a bioelectric stimulator, which is programmed to produce bioelectric signals that cause the patient to upregulate the expression of OPG so as to enhance osteoblast formation and bone formation/re-mineralization for implant stability. In certain embodiments, controlled release of OPG (which promotes osseointegration (bone in growth)) is combined with upregulation of expression of SDF1 and PDGF to promote stem cell homing and healing, which promotes rapid regeneration of tissues.

In certain embodiments, the described methods include use of a bioelectric stimulator, which is programmed to produce a bioelectric signal that causes the patient to upregulate the expression of transforming growth factor beta 1 (TGF-β1). TGF-β1 is a polypeptide member of the transforming growth factor beta superfamily of cytokines, which is a secreted protein that performs many cellular functions, including the control of cell growth, cell proliferation, cell differentiation, and apoptosis. A bioelectric signal that upregulates expression of TGF-β1 in a cell is a square, biphasic waveform at 50% duty, wherein the frequency is at least 75 Hz and the signal amplitude is typically about 1.0 V as measured at the cellular level.

In certain embodiments, rapid accelerated healing in, for example, implant dentistry is attained with combination bioelectric and biologics therapy keyed on highly precise controlled protein expressions, such as OPG, SDF1, PDGF, VEGF, and IGF1, so as to activate, amplify, extend, enhance, and accelerate the natural healing process.

Described is a bioelectric stimulator that is programmed to upregulate expression of OPG, klotho, RANKL, IGF1, and bioelectric PRF combination.

In certain exemplary embodiments, the following sequence of bioelectric signals for promoting rapid dental implant healing and pain reduction is applied to a patient, and includes a bioelectric signal for SDF1 for about 5 minutes, a bioelectric signal for PDGF for about 5 minutes, a bioelectric signal for IGF1 for about 5 minutes, a bioelectric signal for BMP 9 for about 2.5 minutes, a bioelectric signal for OPG for about 5 minutes, a bioelectric signal for klotho for about 5 minutes, and a bioelectric signal for managing inflammation and pain for about 5 minutes. See, e.g., the incorporated U.S. Pat. No. 11,110,274 to Leonhardt (Sep. 7, 2021) for “System and method for treating inflammation” for bioelectric signals useful to manage pain and inflammation.

In certain embodiments, the described bioelectric signals are applied wirelessly to the patient. In certain such embodiments, a micro coil receiver crown is placed on the top of an implanted dental implant, which receiver communicates with an external electromagnetic energy generator that serves to allow the implant to deliver specific bioelectric signals to the surrounding tissues to control release of the specific proteins that accelerate dental implant healing, osseointegration (bone in growth) and reduce associated pain and discomfort.

In certain embodiments (e.g., FIG. 3), a bioelectric mouthpiece is electrically connected to a FDA cleared stimulator, which delivers specific bioelectric signals for modulating (upregulating or downregulating) the expression of proteins selected to accelerate, for example, dental implant healing, osseointegration (bone in growth), and to reduce associated pain and discomfort. For example, when programmed as described herein, the system depicted in FIG. 3 decreases the required time for a dental implant to be fully utilized by a patient in normal activities such as chewing food without pain.

As shown in FIG. 3, in some embodiments, a system includes a housing, an emitter, and an electronic circuit. The housing (or mouthpiece) is configured to fit within a patient's mouth. The emitter is configured to emit an effective amount of bioelectric signals either wirelessly or via wires and conductive electrode contact to the alveolar soft tissue when the apparatus is disposed within the mouth. The electronic circuit is operatively coupled to the bioelectric signaling emitter. The electronic circuit is configured to control the emitter when the housing is disposed within the mouth and the apparatus is in use during treatment. The system and is used for accelerated healing, reduced risk of infection, alleviating inflammation, and pain relief. Specific bioelectric signal sequences cause the release of the selected protein expressions for specific purposes.

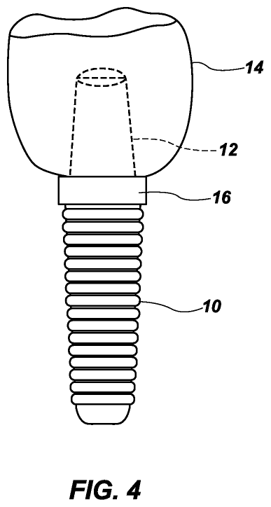

As shown in FIG. 4, after successful implantation, a dental implant typically contains three portions, the post implant 10, the abutment 12, and the crown 14. The post implant 10 is typically a titanium or titanium alloy implant that is surgically fused with the jawbone (e.g., screwed into the jawbone to resemble a tooth root). Other materials such as other metals and ceramics may also be used as an The abutment 12 is typically made of titanium, gold, or porcelain, and fitted over the portion of the post that protrudes from the gum line after the patient's gum tissues are healed. The depicted abutment has an implant abutment interface/implant collar 16.

Initially, the implant is placed or inserted into the bone of the patient's jawline, and a removable cover screw is fitted into the implant abutment interface 16. The healing process of the bone then begins. Without the bioelectric signal therapy described herein, this healing process can take from 3 to 18 months. This healing period is reduced with the application of the described bioelectric signals in the schedule described herein.

As described herein, the described bioelectric signals are applied via electrodes or wirelessly to the post implant 10, the cover screw, and/or the abutment 12 to aid and hasten the healing process. In embodiments where the crown is electrically conductive, the bioelectric signals may alternatively or also be applied to the crown.

After healing, the cover screw is typically removed, and is replaced with the abutment 12. The crown (which is typically a high quality porcelain, metal material, metal fused porcelain, or ceramic resin) is fitted onto the abutment for use and for a natural appearance.

In certain embodiments such as a wireless embodiment, a micro coil receiver crown or micro coil receiver screw is placed on the top of the post implant 10, which micro coil receiver communicates with an external electromagnetic energy generator that serves to induce the implant to deliver specific bioelectric signals to surrounding cellular tissues to modulate expression of the selected proteins so as to accelerate implant healing, osseointegration (bone in growth), and reduction of associated pain and discomfort.

In certain embodiments, the described bioelectric signal therapy is combined with repeat delivery of biologics via a slow infusion re-fillable micro infusion pump placed on top of the dental implants (e.g., incorporated into the crown, abutment, or screw). This micro pump is re-filled daily or weekly with an implant healing acceleration composition comprising a combination selected from the group consisting of tooth pulp stem cells, bioelectric PRF, amniotic fluid, micro RNA gel, bone matrix, selected growth factors such as BMPs, nutrient hydrogel, oxygenated nanoparticles, and bone matrix.

For example, use of the bioelectrically stimulated implant device may be combined with the administration of any or all of these biologic agents: stem cells, selected exosomes, platelet rich fibrin PRF, platelet rich plasma PRP, amniotic fluid, placental fluid, selected growth factors such as OPG and BMP9, nutrient hydrogel, micron RNA gel, Wharton's Jelly, oxygenated nanoparticles, fibrin gel, extracellular matrix and bone matrix.

The invention is further described with the aid of the following illustrative Examples.

### EXAMPLES

### Example I

A trial was conducted as follows. A biostimulator (Mettler) was programmed to produce bioelectric signals that upregulate expression of SDF1, PDGF1, VEGF, IGF1, and OPG by human cells. Controlled expression of OPG promotes osseointegration (bone in growth). SDF1 and PDGF promote stem cell homing and healing, which promotes rapid regeneration of tissues. A first therapy of bioelectric signals was applied to a dental implant patient on day one as follows:
A bioelectric signal for SDF1 for 5 minutes at 5 mA
A bioelectric signal for PDGF1 for 5 minutes
A bioelectric signal for VEGF for 5 minutes
A bioelectric signal for IGF1 for 5 minutes
A bioelectric signal for OPG for 10 minutes

On day three, a series of bioelectric signals was applied to the implant as follows:
A bioelectric signal for SDF1 for 5 minutes at 4 mA to 4.5 mA
A bioelectric signal for PDGF1 for 5 minutes at 4 mA
A bioelectric signal for VEGF for 5 minutes at 2 mA
A bioelectric signal for IGF1 for 5 minutes at 200 μA to 685 μA
A bioelectric signal for OPG for 10 minutes at 3 mA

On day five, a series of bioelectric signals was applied to the implant as follows:
A bioelectric signal for SDF1 for 5 minutes at 3.2 mA to 4 mA
A bioelectric signal for PDGF1 for 5 minutes at 3.6 to 3.2 mA
A bioelectric signal for VEGF for 5 minutes at 2 mA
A bioelectric signal for IGF1 for 5 minutes at 625 μA
A bioelectric signal for OPG for 10 minutes at 3.4 mA

On day seven, a series of bioelectric signals was applied to the implant as follows:
A bioelectric signal for SDF1 for 5 minutes at 3.2 mA to 4.0 mA
A bioelectric signal for PDGF1 for 5 minutes at 2.4 mA
A bioelectric signal for VEGF for 5 minutes at 2 mA
A bioelectric signal for IGF1 for 5 minutes at 645 μA
A bioelectric signal for OPG for 10 minutes at 3.4 mA

On day nine, a series of bioelectric signals was applied to the implant as follows:
A bioelectric signal for SDF1 for 5 minutes at 3.6 mA to 3.2 mA
A bioelectric signal for PDGF1 for 5 minutes at 2.4 mA to 2.2 mA
A bioelectric signal for VEGF for 5 minutes at 2 mA to 1.6 mA
A bioelectric signal for IGF1 for 5 minutes at 645 μA to 600 μA
A bioelectric signal for OPG for 10 minutes at 3.0 mA

On day 11, a series of bioelectric signals was applied to the implant as follows:
A bioelectric signal for SDF1 for 5 minutes at 3.6 mA
A bioelectric signal for PDGF1 for 5 minutes at 2.3 mA
A bioelectric signal for VEGF for 5 minutes at 2 mA to 2.2 mA
A bioelectric signal for IGF1 for 5 minutes at 650 μA
A bioelectric signal for OPG for 10 minutes at 3.0 mA

On day 14, a series of bioelectric signals was applied to the implant as follows:
A bioelectric signal for SDF1 for 5 minutes at 3.7 mA
A bioelectric signal for PDGF1 for 5 minutes at 2.4 mA
A bioelectric signal for VEGF for 5 minutes at 2.2 mA
A bioelectric signal for IGF1 for 5 minutes at 650 μA
A bioelectric signal for OPG for 10 minutes at 3.0 mA

On day 16, a series of bioelectric signals was applied to the implant as follows:
A bioelectric signal for SDF1 for 5 minutes at 3.7 mA
A bioelectric signal for PDGF1 for 5 minutes at 2.4 mA
A bioelectric signal for VEGF for 5 minutes at 2.6 mA
A bioelectric signal for IGF1 for 5 minutes at 650 μA
A bioelectric signal for OPG for 10 minutes at 3.0 mA

On day 19, a series of bioelectric signals was applied to the implant as follows:
A bioelectric signal for SDF1 for 5 minutes at 3.4 mA
A bioelectric signal for PDGF1 for 5 minutes at 2.8 mA
A bioelectric signal for VEGF for 5 minutes at 2.6 mA
A bioelectric signal for IGF1 for 5 minutes at 670 μA
A bioelectric signal for OPG for 10 minutes at 3.6 mA

On day 21, a series of bioelectric signals was applied to the implant as follows:
A bioelectric signal for SDF1 for 5 minutes at 3.5 mA
A bioelectric signal for PDGF1 for 5 minutes at 3.0 mA
A bioelectric signal for VEGF for 5 minutes at 3.0 mA
A bioelectric signal for IGF1 for 5 minutes at 675 μA
A bioelectric signal for OPG for 10 minutes at 3.5 mA

On day 24, a series of bioelectric signals was applied to the implant as follows:
A bioelectric signal for SDF1 for 5 minutes at 3.5 mA
A bioelectric signal for PDGF1 for 5 minutes at 3.0 mA
A bioelectric signal for VEGF for 5 minutes at 2.4 mA
A bioelectric signal for IGF1 for 5 minutes at 670 μA
A bioelectric signal for OPG for 10 minutes at 3.6 mA

On day 26, a series of bioelectric signals was applied to the implant as follows:
A bioelectric signal for SDF1 for 5 minutes at 3.5 mA
A bioelectric signal for PDGF1 for 5 minutes at 3.0 mA
A bioelectric signal for VEGF for 5 minutes at 2.4 mA
A bioelectric signal for IGF1 for 5 minutes at 670 μA
A bioelectric signal for OPG for 10 minutes at 3.6 mA

On day 28, a series of bioelectric signals was applied to the implant as follows:
A bioelectric signal for SDF1 for 5 minutes at 3.6 mA
A bioelectric signal for PDGF1 for 5 minutes at 3.0 mA
A bioelectric signal for VEGF for 5 minutes at 2.4 mA to 2.2 mA
A bioelectric signal for IGF1 for 5 minutes at 675 μA
A bioelectric signal for OPG for 10 minutes at 3.6 mA

On day 30, a series of bioelectric signals was applied to the implant as follows:
A bioelectric signal for SDF1 for 5 minutes at 3.6 mA decreased to 3.5 mA
A bioelectric signal for PDGF1 for 5 minutes at 3.0 mA decreased to 2.8 mA
A bioelectric signal for VEGF for 5 minutes at 2.4 mA
A bioelectric signal for IGF1 for 5 minutes at 675 μA
A bioelectric signal for OPG for 10 minutes at 3.6 mA

On day 32, a series of bioelectric signals was applied to the implant as follows:
A bioelectric signal for SDF1 for 5 minutes at 3.5 mA
A bioelectric signal for PDGF1 for 5 minutes at 2.8 mA
A bioelectric signal for VEGF for 5 minutes at 2.4 mA
A bioelectric signal for IGF1 for 5 minutes at 675 μA
A bioelectric signal for OPG for 10 minutes at 3.6 mA

On day 34, a series of bioelectric signals was applied to the implant as follows:
A bioelectric signal for SDF1 for 5 minutes at 3.6 mA
A bioelectric signal for PDGF1 for 5 minutes at 3.0 mA
A bioelectric signal for VEGF for 5 minutes at 2.4 mA to 2.2 mA
A bioelectric signal for IGF1 for 5 minutes at 675 μA
A bioelectric signal for OPG for 10 minutes at 3.6 mA

On day 36, a series of bioelectric signals was applied to the implant as follows:
A bioelectric signal for SDF1 for 5 minutes at 3.5 mA
A bioelectric signal for PDGF1 for 5 minutes at 2.8 mA
A bioelectric signal for VEGF for 5 minutes at 2.4 mA
A bioelectric signal for IGF1 for 5 minutes at 675 μA
A bioelectric signal for OPG for 10 minutes at 3.6 mA

On day 38, a series of bioelectric signals was applied to the implant as follows:
A bioelectric signal for SDF1 for 5 minutes at 3.5 mA
A bioelectric signal for PDGF1 for 5 minutes at 2.8 mA
A bioelectric signal for VEGF for 5 minutes at 2.4 mA
A bioelectric signal for IGF1 for 5 minutes at 675 μA
A bioelectric signal for OPG for 10 minutes at 3.6 mA

On day 40, a series of bioelectric signals was applied to the implant as follows:
A bioelectric signal for SDF1 for 5 minutes at 3.5 mA
A bioelectric signal for PDGF1 for 5 minutes at 2.8 mA
A bioelectric signal for VEGF for 5 minutes at 2.4 mA
A bioelectric signal for IGF1 for 5 minutes at 675 μA
A bioelectric signal for OPG for 10 minutes at 3.6 mA

On day 42, a series of bioelectric signals was applied to the implant as follows:
A bioelectric signal for SDF1 for 5 minutes at 3.5 mA
A bioelectric signal for PDGF1 for 5 minutes at 2.8 mA
A bioelectric signal for VEGF for 5 minutes at 2.4 mA
A bioelectric signal for IGF1 for 5 minutes at 675 μA
A bioelectric signal for OPG for 10 minutes at 3.6 mA

On day 45, a series of bioelectric signals was applied to the implant as follows:
A bioelectric signal for SDF1 for 5 minutes at 3.6 mA
A bioelectric signal for PDGF1 for 5 minutes at 3.0 mA
A bioelectric signal for VEGF for 5 minutes at 2.5 mA
A bioelectric signal for IGF1 for 5 minutes at 680 μA
A bioelectric signal for OPG for 10 minutes at 3.6 mA

On day 47, a series of bioelectric signals was applied to the implant as follows:
A bioelectric signal for SDF1 for 5 minutes at 3.6 mA
A bioelectric signal for PDGF1 for 5 minutes at 3.0 mA
A bioelectric signal for VEGF for 5 minutes at 2.5 mA
A bioelectric signal for IGF1 for 5 minutes at 680 μA
A bioelectric signal for OPG for 10 minutes at 3.6 mA

On day 49, a series of bioelectric signals was applied to the implant as follows:
A bioelectric signal for SDF1 for 5 minutes at 3.6 mA
A bioelectric signal for PDGF1 for 5 minutes at 3.0 mA
A bioelectric signal for VEGF for 5 minutes at 2.5 mA
A bioelectric signal for IGF1 for 5 minutes at 680 μA
A bioelectric signal for OPG for 10 minutes at 3.6 mA

The patient from the trial experienced enhanced osteointegration of the implant.

### Example II

Four patients needing implants underwent therapy utilizing the described therapy (stimulation twice a week for 40 minutes for 8 weeks). All four patients demonstrated a 300% acceleration in bone osteointegration and implant stabilization time compared to historical control data as measured by an OssTell device. See, e.g., Soicu et al. “An evaluation of TimPlant using Osstell: A device for non-invasive assessment of dental implants stability” OHDMBSC, Vol. VIII. No. 4, pp. 28-33 (December 2009).

### Example III

Osteoblasts were cultured in 6-well plates. Using carbon electrodes inside the culture, biphasic square pulses at frequencies between 20 Hz and 3 MHz were applied. OPG was quantified by OPG mRNA extracted immediately after the stimulation protocol through RTqPCR. Data from stimulated cells was normalized to mRNA from control sister dishes and GAPDH expression. Changes in expression were plotted against frequency.

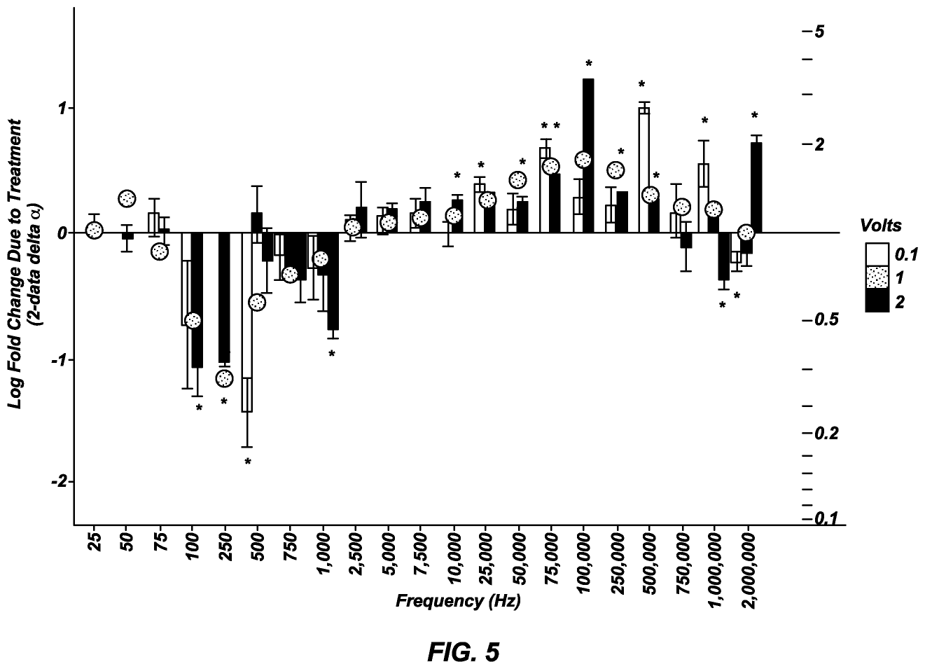

FIG. 5 is a graph depicting cumulative results of OPG expression for the specific bioelectric signal application protocols as applied to osteoblasts. In the graph, * indicates a significant log fold change due to treatment. The right tick marks indicate fold change. The circles indicate predicted values from Generalized Additive Model.

The data (FIG. 5) demonstrates that below 2000 Hz, the expression of OPG mRNA was significantly reduced (70%), with a lowest reached at ˜200 Hz. More importantly, the expression of OPG mRNA was significantly increased at frequencies above 2000 Hz with a maximal expression (200%) at ˜100,000 Hz. This data was consistent whereas the voltage applied was of 0.1, 1, or 2 volts. Overall, the results support that the expression of OPG can be regulated by the frequency of squared pulses at low voltages.

The results depicted in FIG. 5 indicate that there are primarily two regions in the frequency spectrum. A first region that reduces expression OPG by a cell from the basal level of between 75 and 1,000 Hz. The second region corresponds to overexpression of OPG by application of a bioelectric signal of between 2,500 Hz and 3,000,000 Hz.

### Example IV

A cell was treated by stimulating the cell with a bioelectric signal so as to upregulate expression of transforming growth factor beta 1 (TGF-β1) by the cell. The bioelectric signal stimulation was by applying a bioelectric signal to the cell, wherein the bioelectric signal was a square, biphasic waveform at 50% duty, wherein the frequency was 75 Hz and the signal amplitude was about 1.0 V as measured at the cell. The cell experienced upregulated expression of TGF-β1.

### REFERENCES

(the contents of the entirety of each of which is incorporated herein by this reference):
Bhatt et al. “MicroRNA-687 Induced by Hypoxia-Inducible Factor-1 Targets Phosphatase and Tensin Homolog in Renal Ischemia-Reperfusion Injury” JASN July 2015, 26 (7) 1588-1596; DOI: doi.org/10.1681/ASN.2014050463.
Cerrada et al. “Hypoxia-inducible factor 1 alpha contributes to cardiac healing in mesenchymal stem cells-mediated cardiac repair” Stem Cells Dev. 2013 Feb. 1; 22(3):501-11. doi: 10.1089/scd.2012.0340. Epub 2012 Sep. 14. PMID: 22873764.
do Nascimento C, Issa J P, Mello A S, de Albuquerque Junior R F. “Effect of electromagnetic field on bone regeneration around dental implants after immediate placement in the dog mandible: a pilot study.” Gerodontology. 2012 June; 29(2):e1249-51. doi: 10.1111/j.1741-2358.2011.00525.x. PMID: 22612842.
“Electrical stimulation speeds bone graft healing and can increase success of grafting for dental implants” PERIO-IMPLANT Advisory, www.perioimplantadvisory. com/clinical-tips/surgical-techniques/article/16412179/electrical-stimulation-speeds-bone-graft-healing-and-can-increase-success-of-grafting-for-dental-implants (Feb. 23, 2012).
Guimaraes-Camboa, N. and Evans, S. “Redox Paradox: Can Hypoxia Heal Ischemic Hearts?” Developmental Cell 39(4): 392-394 (Nov. 21, 2016); DOI: doi.org/10.1016/j.devce1.2016.11.007.
Jin, Qiming, and William V Giannobile. “SDF-1 enhances wound healing of critical-sized calvarial defects beyond self-repair capacity.” PloS one vol. 9.5 e97035. 6 May. 2014, doi:10.1371/journal.pone.0097035.
Lin et al. “Hypoxia-Inducible Factor 2 Alpha Is Essential for Hepatic Outgrowth and Functions via the Regulation of leg1 Transcription in the Zebrafish Embryo” PLoS ONE 9(7): e101980; doi.org/10.1371/journal.pone.0101980.
Liu, Yiming et al. “The effect of osteoprotegerin on implant osseointegration in ovariectomized rats.” Archives of Medical Science: AMS vol. 13.2 (2017): 489-495; doi:10.5114/aoms.2017.65468.
Nath et al. “Hypoxia and hypoxia inducible factors: diverse roles in liver diseases.” Hepatology (Baltimore, Md.) vol. 55, 2 (2012): 622-33. doi:10.1002/hep.25497.
Soicu et al. “An evaluation of TimPlant using Osstell: A device for non-invasive assessment of dental implants stability” OHDMBSC, Vol. VIII. No. 4, pp. 28-33 (December 2009).
Tajima et al. “HIF-1alpha is necessary to support gluconeogenesis during liver regeneration.” Biochem Biophys Res Commun. 2009 Oct. 2; 387(4):789-94. doi: 10.1016/j.bbrc.2009.07.115. Epub 2009 Jul. 28. PMID: 19643083.
Wig-Bürgel “Hypoxia-Inducible Factor-1 (HIF-1): A Novel Transcription Factor in Immune Reactions” Journal Of Interferon & Cytokine Research 25:297-310 (2005).
Zhou et al. Nerve electrical stimulation enhances osseointegration of implants in the beagle. Sci Rep 9, 4916 (2019). doi.org/10.1038/s41598-019-41471-z.
Zutshi, et al. “Low Voltage Electrical Stimulation Causes Cytokine Expression in the Anal Sphincter” 770-771 (2009).
US Patent Application Publication US 20200000709 A1 to Leonhardt et al. (Jan. 2, 2020) for “Combination of Bioelectrical Stimulator and Platelet-Rich Fibrin for Accelerated Healing and Regeneration.”
US Patent Application Publication US 20200324106 A1 to Leonhardt et al. (Oct. 15, 2020) for “Bioelectric Stimulation for Sonic Hedgehog Expression.”
US Patent Application Publication US 20200330759 A1 to Leonhardt et al. (Oct. 22, 2020) for “Bioelectric OPG Treatment of Cancer.”
US Patent Application Publication US 20200330753 A1 to Leonhardt et al. (Oct. 22, 2020) for “Orthodontic Treatment.”
U.S. Pat. No. 8,909,346 B2 to Chalmers (Dec. 9, 2014) for “Frequency Specific Microcurrent for Treatment of Dental Indications.”
U.S. Pat. No. 10,646,644 B2 to Leonhardt et al. (May 12, 2020) for “Stimulator, pump and composition.”
U.S. Pat. No. 10,695,563 B2 to Leonhardt et al. (Jun. 30, 2020) for “Orthodontic treatment.”
U.S. Pat. No. 10,960,206 to Leonhardt et al. (Mar. 30, 2021) for “Bioelectric Stimulator.”
U.S. Pat. No. 11,110,274 to Leonhardt (Sep. 7, 2021) for “System and method for treating inflammation.”
US Patent Application Publication US 20200000709 A1 to Leonhardt et al. (Jan. 2, 2020) for “Combination of bioelectrical stimulator and platelet-rich fibrin for accelerated healing and regeneration.”
US Patent Application Publication US 2020-0289826-A1 to Leonhardt et al. (Sep. 17, 2020) for “Klotho Modulation.”
U.S. patent application Ser. No. 17/473,809 to Leonhardt, filed Sep. 13, 2021.
U.S. Patent Application Ser. No. 63/215,841 (filed Jun. 28, 2021) to Leonhardt et al. for “Modulation of Vascular Endothelial Growth Factor (VEGF) and Pulse Width Utilization.”
U.S. Provisional Patent Application Ser. No. 63/189,586 (filed May 17, 2021) to Leonhardt et al. for “BMP9 Modulation.”

1. A method of accelerating the healing of an implant in a subject, the method comprising:
applying to the implant a bioelectric signal selected from the group consisting of a biphasic pulse of between 2,500 Hz and 750,000 Hz and a biphasic pulse of about 3,000,000 Hz, with a voltage of between 0.001 volts and 4 volts, so as to promote bone osteointegration of the implant.

2. The method according to claim 1, further comprising:
stimulating the implant by applying a bioelectric signal to the implant to upregulate expression of bone morphogenic protein 9 (BMP9), wherein the bioelectric signal is a biphasic pulse of between 100 Hz and 300 Hz with a voltage of between 0.1 volts and 4 volts as measured at the cell.

3. A method of accelerating the healing of an implant in a subject, the method comprising:
applying to the implant a bioelectric signal selected from the group consisting of a biphasic pulse of between 2,000 Hz and 750,000 Hz and a biphasic pulse of about 3,000,000 Hz, with a voltage of between 0.001 volts and 4 volts, and
stimulating the implant by applying a bioelectric signal to the implant to upregulate expression of bone morphogenic protein 9 (BMP9), wherein the bioelectric signal is a biphasic pulse of between 100 Hz and 300 Hz with a voltage of between 0.1 volts and 4 volts as measured at the cellular level,
so as to promote bone osteointegration of the implant.

4. A method of treating a cell, the method comprising:
stimulating the cell to modulate expression of hypoxia-inducible factor 1-alpha (“HIF1α”) by the cell by applying a bioelectric signal to the cell, wherein the bioelectric signal is selected from the group consisting of 0.25 mA to 0.75 mA (3.0 V), 80 to 100 Hz, 80 to 110 μs pulse width, square wave and 30 Hz, 3.5 mV, with the voltage measured at the cell.

5. The method according to claim 4, wherein the bioelectric signal is 0.25 mA to 0.75 mA (3.0V), 80 to 100 Hz, 80 to 110 μs pulse width, square wave, and HIF1α expression is upregulated.

6. The method according to claim 4, wherein the bioelectric signal is 30 Hz, 3.5 mV, and HIF1α expression is downregulated.

7. A method of accelerating the healing of a bone graft in a subject, the method comprising:
applying to the bone graft and cellular tissue surrounding the bone graft a bioelectric signal selected from the group consisting of a biphasic pulse of between 2,000 Hz and 750,000 Hz and a biphasic pulse of about 3,000,000 Hz, with a voltage of between 0.001 volts and 4 volts, and
stimulating the implant by applying a bioelectric signal to the implant to upregulate expression of bone morphogenic protein 9 (BMP9), wherein the bioelectric signal is a biphasic pulse of between 100 Hz and 300 Hz with a voltage of between 0.1 volts and 4 volts as measured at the cellular level.

8. A method of treating a cell, the method comprising:
stimulating the cell to upregulate expression of osteoprotegerin (OPG) by the cell by applying a bioelectric signal to the cell, wherein the bioelectric signal is selected from the group consisting of a biphasic pulse of between 2,500 Hz and 750,000 Hz and a biphasic pulse of about 3,000,000 Hz, with a voltage of between 0.001 volts and 4 volts as measured at the cell.

9. The method according to claim 8, wherein the bioelectric signal is a biphasic pulse of between 10,000 Hz and 500,000 Hz with a voltage of between 0.1 volts and 2 volts as measured at the cell.

10. The method according to claim 8, further comprising:
stimulating the cell to upregulate expression of bone morphogenic protein 9 (BMP9) by applying a bioelectric signal to the cell, wherein the bioelectric signal is a biphasic pulse of between 100 Hz and 300 Hz with a voltage of between 0.1 volts and 4 volts as measured at the cell.

11. The method according to claim 8, wherein the cell is comprised within a subject.

12. The method according to claim 11, wherein the subject is undergoing a bone graft.

13. The method according to claim 8, wherein the stimulation causes OPG release to enhance osteoblast formation and bone formation/re-mineralization to increase stability of a dental implant.

14. The method according to claim 8, further comprising:
Applying a bioelectric signal of a continuous current of 10 μA (as measured at the cellular level), for 5 minutes, where the continuous current has a biphasic waveform, with a frequency of 50 Hz.

15. A method of treating a cell, the method comprising:
stimulating the cell to downregulate expression of osteoprotegerin by the cell by applying a bioelectric signal to the cell, wherein the bioelectric signal is selected from the group consisting of a biphasic pulse of between 100 Hz and 1,000 Hz and a biphasic pulse of about 1,000,000 Hz, with a voltage of between 0.001 volts and 4 volts as measured at the cell.

16. The method according to claim 15, wherein the bioelectric signal is a biphasic pulse of between 100 Hz and 500 Hz with a voltage of between 0.1 volts and 2 volts as measured at the cell.

17. The method according to claim 15, wherein the cell is comprised within a subject.

18. A method of treating a cell, the method comprising:
stimulating the cell to upregulate expression of transforming growth factor beta 1 (TGF-β1) by the cell by applying a bioelectric signal to the cell, wherein the bioelectric signal is a square, biphasic waveform at 50% duty, wherein the frequency is at least 75 Hz and the signal amplitude is about 1.0 V as measured at the cell.
